(19) United States
(12) Patent Application Publication
(10) Pub. No.: US 2013/0315849 A1
(43) Pub. Date: Nov. 28, 2013

(54) USE OF 13-HYDROXY-9,11-OCTADECADIENOIC ACID FOR TANNING HUMAN SKIN

(71) Applicants: Mike Farwick, Essen (DE); Tim Koehler, Dorsten (DE)

(72) Inventors: Mike Farwick, Essen (DE); Tim Koehler, Dorsten (DE)

(73) Assignee: EVONIK GOLDSCHMIDT GMBH, Essen (DE)

(21) Appl. No.: 13/976,204

(22) PCT Filed: Dec. 12, 2011
(86) PCT No.: PCT/EP2011/072033

§ 371 Date: Jun. 26, 2013

(30) Foreign Application Priority Data
Dec. 30, 2010 (DE) 102010056550.4

(51) Int. Cl. A61K 8/365; A61Q 17/04; A61Q 5/00

(52) U.S. Cl. CPC A61K 8/365; A61Q 5/00; A61Q 17/04
USPC 424/60; 424/59; 424/70.6

(57) ABSTRACT

The invention relates to the use of 13-hydroxy-9,11-octadecadienoic acid for tanning human skin and methods associated therewith.

### FIELD OF THE INVENTION

The invention relates to the use of 13-hydroxy-9,11-octadecadienoic acid for tanning human skin and to methods associated therewith.

### PRIOR ART

Many consumers would like a darker complexion (“healthy tan”) and therefore either subject themselves to harmful UV radiation or resort to cosmetic or dermatological preparations in the form of so-called “self-tans”.

Artificial skin tanning can be effected by a cosmetic or medicinal route, in which case the following approaches essentially play a role:

By regularly taking carotene preparations, carotene is stored in the subcutaneous fat, and the skin gradually turns orange to yellow-brown.

With the help of wash-off make-up preparations it is possible to achieve a light skin tint (e.g. extracts from fresh green walnut shells, henna).

Colouring can also take place by the route of chemical modification of the horny layer of the skin using so-called self-tanning preparations. The most important active ingredient is dihydroxyacetone (DHA). The skin tan achieved in this way cannot be washed off and is only removed with the normal desquamation of the skin (after ca. 10-15 days). Dihydroxyacetone can be referred to as ketotriose and reacts as a reducing sugar with the amino acids in the skin and/or the free amino and imino groups of keratin via a series of intermediates in the sense of a Maillard reaction to give brown-coloured substances, so-called melanoids, which are also sometimes called melanoidins.

Disadvantages of tanning with dihydroxyacetone is that the skin tanned therewith
is often unevenly tanned
does not have a natural skin tan shade
has an unpleasant smell following application to the skin
it leads to severe discolourations of the palms of the hand and of the nails and
in contrast to “sun-tanned” skin, it is not protected against sunburn.

Dihydroxyacetone often leads to the discolouration of clothes, bed linen and other textiles.

Another way of tanning the skin without radiation is to apply an active ingredient to the skin which stimulates the synthesis of melanin and so achieves tanning as a result of stimulation of the skin's own tanning system. The best known stimulators of melanin synthesis are tyrosine and tyrosine acyl derivatives, in particular N-acetyltyrosine and N-caproyltyrosine and salts thereof. The synthesis of melanin can also be stimulated by D-quiroinositol. The stimulation of the melanin synthesis mimics the natural tanning process and therefore leads to a more natural shade than dihydroxyacetone.

All classical self-tanning products exhibit only very poor skincare properties.

Essential fatty acids have been used for a long time as cosmetic active ingredients. They occur for example in evening primrose oil, grapeseed oil, wheat germ oil, linseed oil, rosehip, currant or raspberry seed oil. These oils contain inter alia linoleic acid (omega-6). This is converted in the skin by the 15-lipoxygenase (15-LOX) and subsequent reduction into a highly effective antiinflammatory metabolite, the antiinflammatory 13-NODE (13-hydroxy-9,11-octadecadienoic acid). 13-HODE inhibits the inflammation-triggering leukotriene LTB4.

Consequently, the topical application of the aforementioned oils and also of 13-NODE itself is described in the literature for reducing dry, sensitive and flaky skin.

Thus, for example, WO2004034958 describes the use of fatty acid derivatives in cosmetic and pharmaceutical preparations for the synthesis of 13-NODE in the epidermis in order to prevent dry skin.

WO2009153169 describes a derivative of 13-hydroxy-9,11-octadecadienoic acid, namely 8-methoxy-13-hydroxy-9,11-octadecadienoic acid, as skin-lightening active ingredient and its use in compositions.

It was an object of the invention to provide a self-tanning agent which is able to overcome at least one disadvantage of the prior art.

### DESCRIPTION OF THE INVENTION

Particularly against the disclosure of WO2009153169, it has been entirely surprising to find that 13-hydroxy-9,11-octadecadienoic acid can be used as active ingredient for tanning the skin.

It is known that the expression of the POMC gene in keratinocytes is activated by UV radiation. POMC is the precursor of α-MSH, a main activator of melanocytes. Increased α-MSH activity induces the tanning reaction of the skin. Unexpectedly and surprisingly, it has now been found that the incubation of keratinocytes with 13-HODE significantly increases the expression of POMC both after UV stimulation and also without prior UV stimulation, which leads to an increased tanning reaction of the skin.

The present invention therefore provides the use of 13-hydroxy-9,11-octadecadienoic acid for tanning human skin, the use of 13-hydroxy-9,11-octadecadienoic acid for stimulating the formation of melanin and methods for tanning or improving the tanning of human skin.

The invention further provides a hair colouring method.

One advantage of the present invention is that 13-hydroxy-9,11-octadecadienoic acid is also suitable for treating the scalp in order to act there on the melanocytes, which bring about the colouration of the regrowing hair, as a result of which the natural hair colour can be produced again.

Further advantages of 13-HODE are that it increases the tanning of the skin, permits an even and natural tanning of the skin, does not develop any undesired odours upon application to the skin, is not associated with undesired drying of the skin, does not cause any undesired discolouration of certain areas of skin, such as e.g. the palms of the hands and the nails, and avoids a discolouration of textiles following application to the skin.

It is also an advantage that a more intensive tanning by sunlight is facilitated despite a high UV photoprotective filter.

Beyond this, a long-lasting skin tan with a more natural colouring is achieved through the use of 13-NODE.

The high storage stability in preparations is particularly advantageous.

In connection with the present invention, the terms “13-hydroxy-9,11-octadecadienoic acid” and “13-HODE”, which are used here synonymously, include all configurational isomers and also stereoisomers of the compound; these are for example 13-hydroxy-9Z,11E-octadecadienoic acid, 13-hydroxy-9E,11Z-octadecadienoic acid, 13-hydroxy-9Z,11Z-octadecadienoic acid, 13-hydroxy-9E,11E-octadecadienoic acid

Unless stated otherwise, all of the percentages (%) given are percentages by mass

In connection with the present invention, preference is given to a 13-HODE which has a 9-Z11-E configuration isomerism.

A 13-HODE which has a S stereoconfiguration on the C13 is advantageous in accordance with the invention.

A 13-HODE which has a 9Z,11E configuration isomerism and S stereoconfiguration on the C13 is particularly preferred.

The present invention provides the use of 13-hydroxy-9,11-octadecadienoic acid as active self-tanning agent in a formulation, in particular in a self-tan or a sunscreen formulation.

Sunscreen formulations are recognized by the person skilled in the art from their obvious preparation per se. The obvious preparation of a sunscreen formulation can be made out in particular from the fact that UV radiation-absorbing substances are present. Examples of such substances are esters of cinnamic esters, ester of salicylic acid, benzophenone, finely disperse metal oxides and salts such as titanium dioxide or zinc oxide.

The present invention further provides the use of 13-hydroxy-9,11-octadecadienoic acid for maintaining an existing skin tan. The term “maintaining an existing skin tan” is to be understood as meaning that skin of a certain degree of tanning lightens less rapidly (loses its tan) over a period under consideration than the same observed skin without further treatment.

The formulations specified in connection with the use according to the invention of 13-NODE are in particular cosmetic formulations. The statements made hereinbelow with regard to “formulations” are likewise applicable for the formulations used in the method according to the invention described below and also refer to these.

These formulations comprise preferably from 0.00001 percent by mass to 1 percent by mass, preferably 0.00005 percent by mass to 0.5 percent by mass, particularly preferably 0.0001 percent by mass to 0.1 percent by mass, of 13-hydroxy-9,11-octadecadienoic acid, based on the total mass of the formulation.

The formulation used in the methods according to the invention can comprise e.g. at least one additional component selected from the group of

emollients,

emulsifiers,

thickeners/viscosity regulators/stabilizers,

UV photoprotective filters,

antioxidants,

hydrotropes (or polyols),

solids and fillers,

pearlescent additives,

deodorant and antiperspirant active ingredients,

insect repellents,

self-tanning agents,

preservatives,

conditioners,

perfumes,

dyes,

cosmetic active ingredients,

care additives,

superfatting agents,

solvents.

Substances which can be used as exemplary representatives of the individual groups are known to the person skilled in the art and can be found for example in the German application DE 102008001788.4. This patent application is hereby incorporated by reference and thus forms part of the disclosure.

As regards further optional components and the amounts used of these components, reference is made expressly to the relevant handbooks known to the person skilled in the art, e.g. K. Schrader, “Grundlagen and Rezepturen der Kosmetika [Fundamentals and formulations of cosmetics]”, 2nd edition, page 329 to 341, Hüthig Buch Verlag Heidelberg.

The amounts of the particular additives are governed by the intended use. Typical guide formulations for the respective applications are known prior art and are contained for example in the brochures of the manufacturers of the particular basic materials and active ingredients. These existing formulations can generally be used without change. If necessary, for the purposes of adaptation and optimization, the desired modifications, however, can be undertaken without complications by means of simple experiments.

Preferably, the formulations in connection with the present invention comprise at least one self-tanning agent and/or a UV photoprotective filter as additional component.

The present invention further provides a non-therapeutic, cosmetic method for tanning human skin without the action of UV radiation, characterized in that 13-hydroxy-9,11-octadecadienoic acid and/or at least one formulation comprising 13-hydroxy-9,11-octadecadienoic acid is applied to the skin.

The action mechanism of 13-hydroxy-9,11-octadecadienoic acid also permits an advantageous tanning of the human skin with UV light exposure since it intensifies the tanning effect of a defined amount of light. Consequently, the present invention likewise provides a non-therapeutic cosmetic method for accelerating and/or intensifying the UV-induced tanning of human skin, characterized in that 13-hydroxy-9,11-octadecadienoic acid and/or at least one formulation comprising 13-hydroxy-9,11-octadecadienoic acid is applied to the skin.

In the same way, the action mechanism of 13-hydroxy-9,11-octadecadienoic acid has proven to be advantageous for alternative tanning methods since, by virtue of this, disadvantages of the alternative methods, such as, for example, uneven colouration, can be corrected.

The present invention therefore further provides a non-therapeutic, cosmetic method for improving the tanning of human skin induced by dihydroxyacetone and/or erythrulose, characterized in that 13-hydroxy-9,11-octadecadienoic acid and/or at least one formulation comprising 13-hydroxy-9,11-octadecadienoic acid is applied to the skin.

The action mechanism of 13-hydroxy-9,11-octadecadienoic acid permits a restoration of the original hair colour of hair that has gone grey as a result of ageing. Consequently, the present invention further provides a non-therapeutic cosmetic method for colouring human hair, preferably human head hair, characterized in that 13-hydroxy-9,11-octadecadienoic acid and/or at least one formulation comprising 13-hydroxy-9,11-octadecadienoic acid is applied to the skin, preferably the scalp.

A common aspect of the aforementioned methods is that although a single application exhibits an effect, in order to achieve long-lasting colourations, a repeated application is necessary; consequently, in the case of the methods according to the invention, the 13-hydroxy-9,11-octadecadienoic acid and/or the at least one formulation comprising 13-hydroxy-9,11-octadecadienoic acid are preferably applied repeatedly, but at least once, in particular twice or three times, daily to the skin.

Like beta-carotene, 13-hydroxy-9,11-octadecadienoic acid can help to prevent or to alleviate polymorphous photodermatosis. In this connection, the treatment of the skin to be protected with 13-NODE leads, via a dual mechanism, to a prevention and/or reduction in the skin change caused by the polymorphous photodermatosis. Firstly, the enhanced melanin production brings about a more efficient protection against the UV radiation of sunlight, which is considered to be a main cause of the symptoms of polymorphous photodermatosis. Secondly, 13-HODE has an anti-inflammatory effect, which leads to skin calming and reducing the negative accompanying symptoms of polymorphous photodermatosis (reddening, itching, blistering etc.).

Consequently, the present invention further provides 13-hydroxy-9,11-octadecadienoic acid for protecting the skin against and/or for treating polymorphous photodermatosis.

The present invention is described by way of example in the examples listed below without any intention of limiting the invention, the scope of application of which arises from the entire description and the claims, to the embodiments specified in the examples.

The following figures form part of the examples:

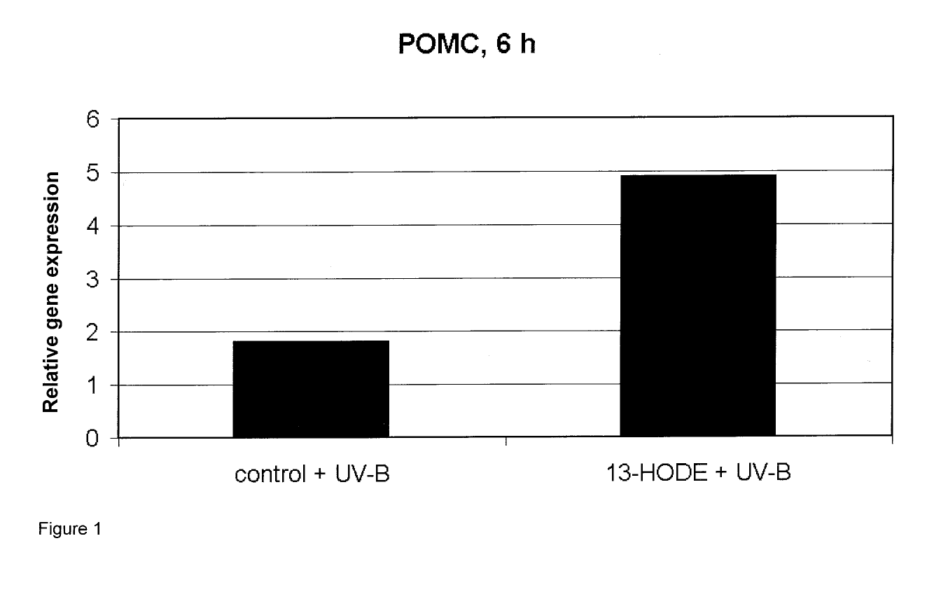

FIG. 1: Stimulation of the UV-induced POMC expression in NHEKs by 13-NODE. The relative gene expression strength of POMC standardized to the 18S rRNA as reference is shown. The values refer to the gene expression strength compared to the control treated only with the vehicle.

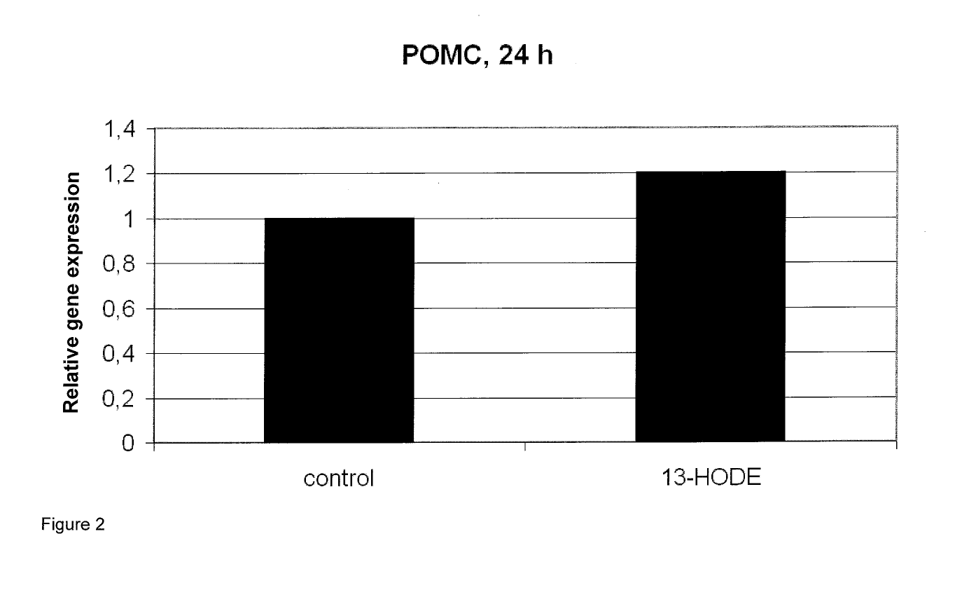

FIG. 2: Stimulation of the basal (i.e. not UV-induced) POMC expression in NHEKs by 13-NODE. The relative gene expression strength of POMC standardized to the 18S rRNA as reference is shown. The values refer to the gene expression strength compared to the control treated only with the vehicle.

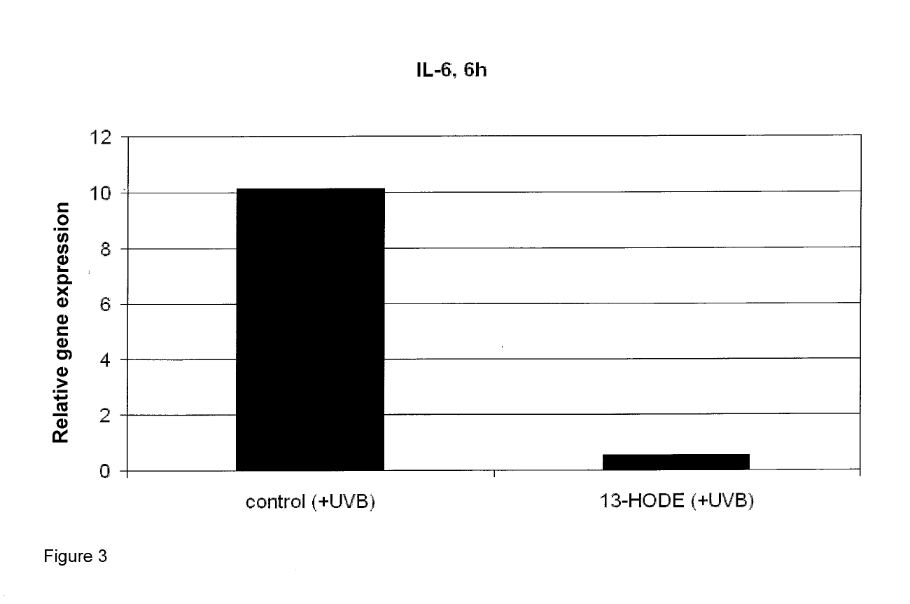

FIG. 3: Reduction in the UV-stimulated induction of IL-6 by 13-HODE. The relative gene expression strength of IL-6 standardized to the 18S rRNA as reference is shown. The bars show the relative induction of the gene expression strength compared to the control treated only with the vehicle.

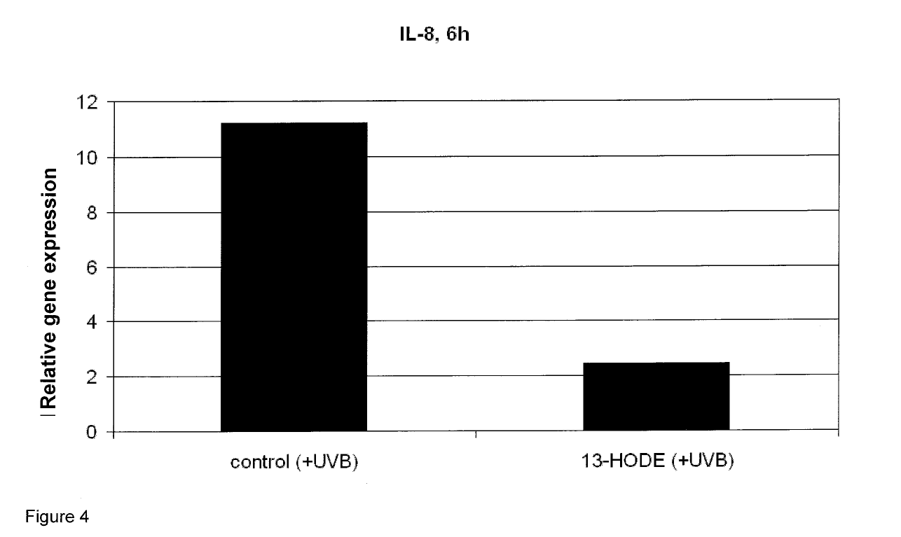

FIG. 4: Reduction in the UV-stimulated induction of IL-8 by 13-NODE. The relative gene expression strength of IL-8 standardized to the 18S rRNA as reference is shown. The bars show the relative induction of the gene expression strength compared to the control treated only with the vehicle.

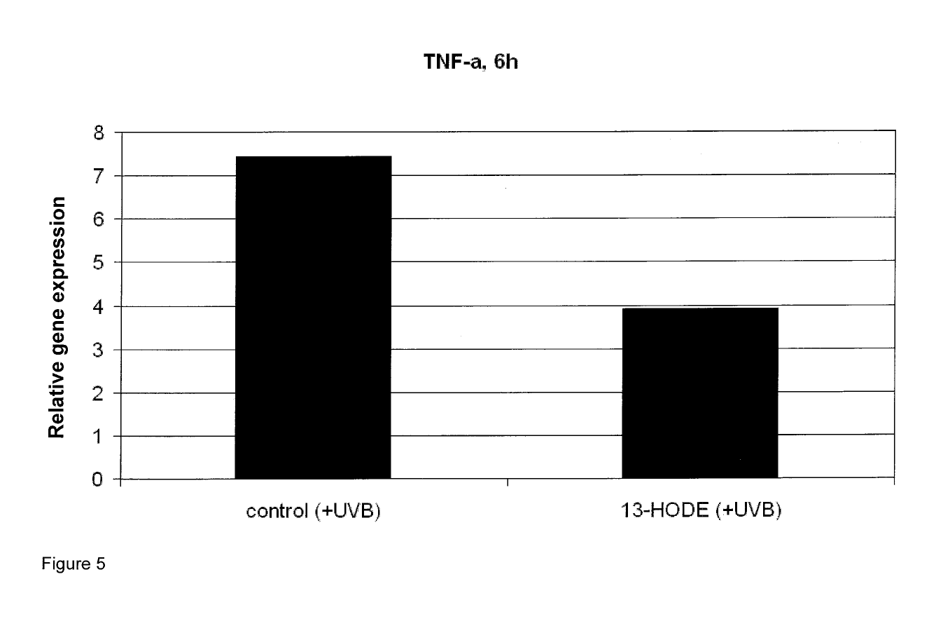

FIG. 5: Reduction in the UV-stimulated induction of TNFα by 13-HODE. The relative gene expression strength of TNFα standardized to the 18S rRNA as reference is shown. The bars show the relative induction of the gene expression strength compared to the control treated only with the vehicle.

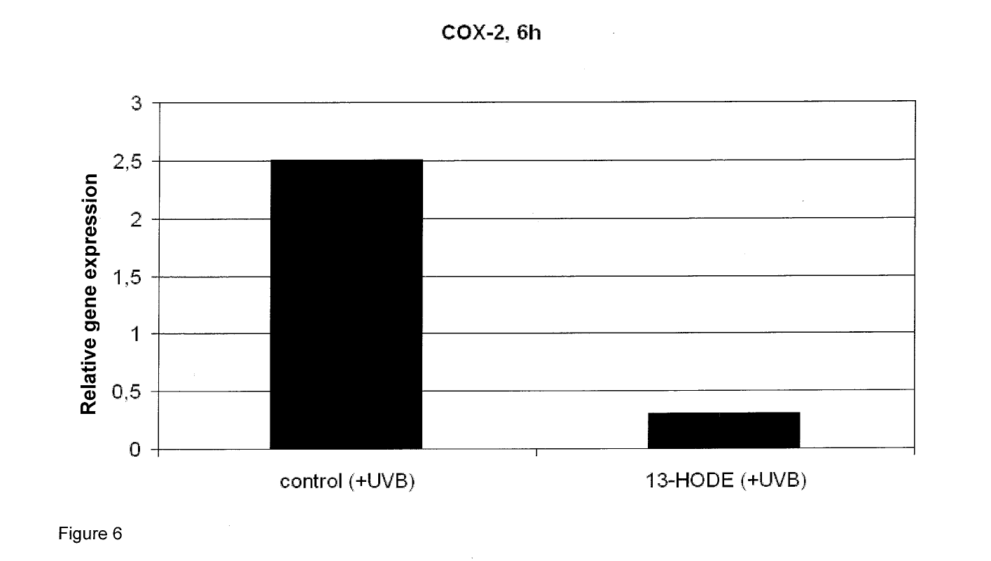

FIG. 6: Reduction in the UV-stimulated induction of COX2 by 13-HODE. The relative gene expression strength of COX2 standardized to the 18S rRNA as reference is shown. The bars show the relative induction of the gene expression strength compared to the control treated only with the vehicle.

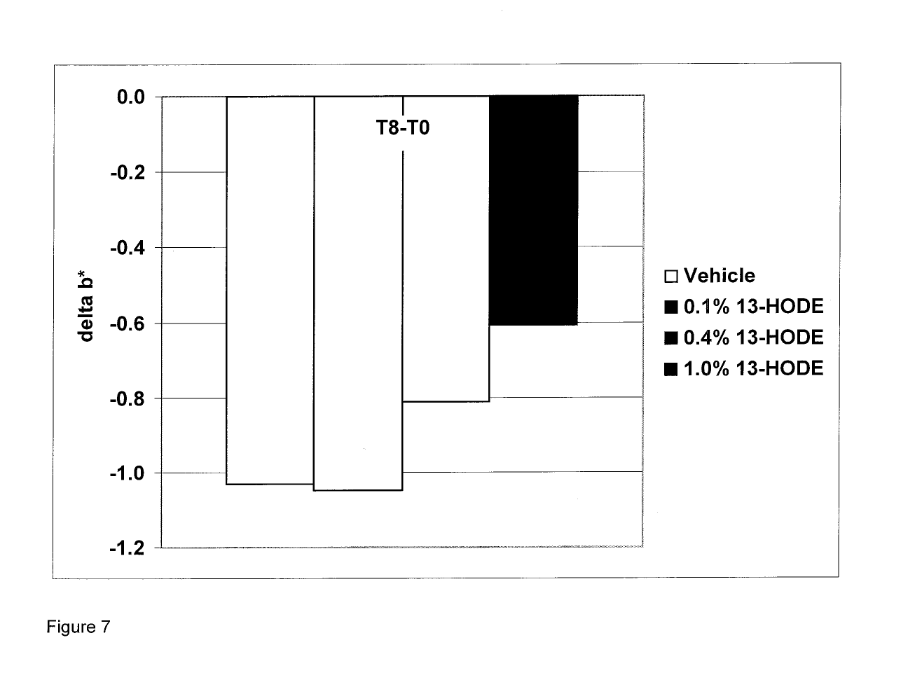

FIG. 7: Blue-yellow parameter b*.

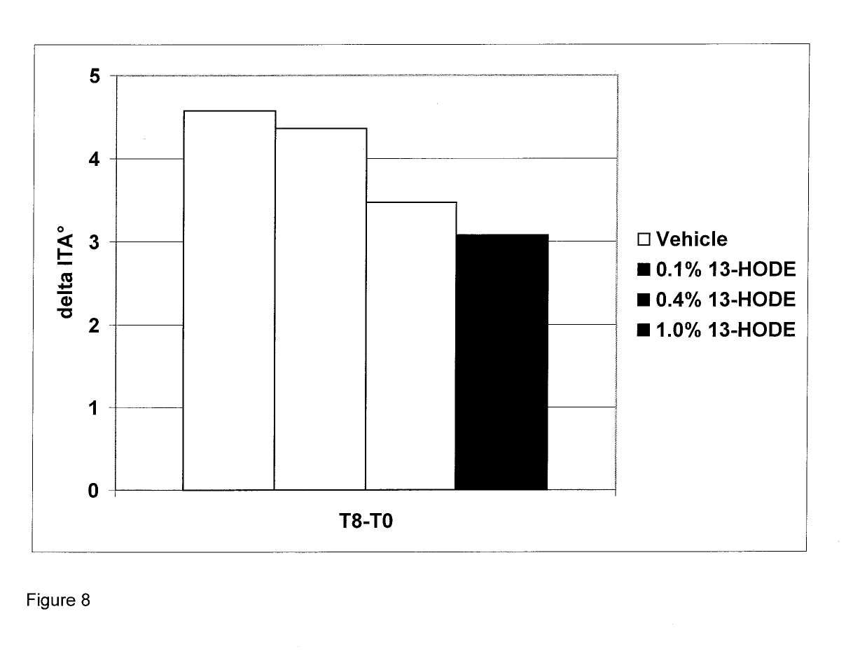

FIG. 8: ITA° (Individual Typology Angle).

### EXAMPLES

### Example 1

### 13-HODE-Stimulated POMC Induction

In the present example, the effect of 13-HODE on the POMC gene expression in UV-B-stimulated keratinocytes (normal human epidermal keratinocytes, NHEKs) was investigated. For this purpose, firstly primary human epidermal keratinocytes were prepared from neonatal foreskin. The procedure is described in the following publications: Grether-Beck et al., J Invest Dermatol 2005, 125: 545-553; and Grether-Beck et al., Exp Dermatol 2008, 17: 771-779. Subsequently, the cells were cultured in defined serum-free keratinocyte growth medium SFM (Invitrogen, Heidelberg, Germany) with the addition of bovine pituitary extract (Invitrogen, Heidelberg, Germany) and recombinant epidermal growth factor (Invitrogen, Heidelberg, Germany). The cells were incubated until passage 2 or 3 at 37° C. and 5% CO₂. For the test as to stimulation of the UV-B-induced POMC gene expression, the cells were sown in 6-well plates and cultured to the point of subconfluence (maximum 70%).

Then, the medium was removed from the cells and replaced by fresh medium supplemented with 13-HODE. For this, a 1000× stock solution of 3000 μg/ml 13-NODE dissolved in DMSO was used.

End concentration of 13-NODE in the medium was 3 μg/ml, end concentration of DMSO in the medium 0.1% (v/v). Cultivations without 13-HODE, only with the vehicle DMSO were carried out as a control. All of the cultivations were undertaken three times (3 biological replicates).

After cultivation for 24 hours, the cells were exposed to a dose of 160 J/m² of UV-B radiation. For this, firstly the medium was replaced by PBS buffer, the lids of the 6-well plates were removed, and the cells were exposed to the radiation under a bench equipped with 4 FS 20 sunlight bulbs (Westinghouse Electric Corporation, Pittsburgh, Pa., USA). The emitted power was determined using an IL 1700 Research Radiometer and SEE 240 UV-B photodetector (International Light Inc., Newbury Port, Mass.) and was 2.4 W/m² at a distance of 22 cm. For comparison purposes, unexposed control batches were also performed alongside in order to be able to ascertain the influence of the UV radiation on the POMC gene expression.

After the exposure, the PBS was replaced by culture medium with 3 μg/ml of 13-HODE, and the cells were cultured for a further 6 h until the point of cell harvest.

Isolation of whole-RNA and subsequent analysis of the gene expression by means of quantitative Real Time PCR (qRT-PCR) was carried out as described previously (Grether-Beck et al., J Invest Dermatol 2005, 125: 545-553; Grether-Beck et al., Exp Dermatol 2008, 17: 771-779). In summary, the whole-RNA was isolated by means of RNeasy Total RNA Kit (Qiagen, Hilden, Germany). The RNA concentration was determined by means of photometric measurement at 260/280 nm (Biophotometer, Eppendorf, Germany). From each sample, 100 ng of whole-RNA were used for the cDNA synthesis, the Superscript®III-First Strand Synthesis System for RT-PCR (Invitrogen, Karlsruhe, Germany) being used. For the PCR reaction, the SYBR® Green PCR Master Mix was used in accordance with the manufacturer's instructions. Besides the POMC expression, the expression of the 18s rRNA gene was also determined quantitatively as a reference. The following primer pairs were used in each case for both target genes:

POMC:
(Seq ID No. 1)
5-′CGCTACGGCGGTTTCATG-3′
and
(Seq ID No. 2)
5-′TGATGATGGCGTTTTTGAACA-3′;
18S:
(Seq ID No. 3)
5′-GCCGCTAGAGGTGAAATTCTTG-3′
and
(Seq ID No. 4)
5′-CATTCTTGGCAAATGCTTTCG-3′.

The PCR reactions were carried out on an Opticon 1 (MJ Research, Waltham, Mass., USA). For all PCRs, double determinations of the biological triplicates were carried out, and for the evaluation averages of all measurements were calculated. The PCR protocol had the following profile: step 1), 10 minute activation of the hot start polymerase at 94° C.; step 2), 20 second denaturation at 95° C.; step 3), 20 second primer annealing at 55° C.; step 4), 30 second extension at 72° C. The cycle number of steps 2)-4) was 46. For the relative comparison of the gene expression, the 2(-delta delta C(T)) method was used (Livak KJ, and Schmittgen TD: Analysis of relative gene expression data using real time quantitative PCR and the 2 (-delta delta C(T)) method. Methods 2001, 25: 402-408).

The evaluation of the gene expression analysis showed that the treatment of the keratinocytes with 13-HODE stimulated the UV-induced POMC expression significantly (p<0.01). Only cells treated with UV exhibited an induction of the POMC expression by a factor of 1.8 compared to the untreated control. By contrast, the stimulation with 13-HODE led in the UV-treated cells to a POMC expression increased by a factor of 4.9 compared to the untreated control.

### Example 2

### 13-HODE-Stimulated POMC Induction without UV

In the present example, the effect of 13-HODE on the POMC gene expression was investigated in keratinocytes which, in contrast to Example 1, have not been stimulated by prior UV exposure. For this purpose, firstly primary human epidermal keratinocytes were prepared from neonatal foreskin. The procedure is described in the following publications: Grether-Beck et al., J Invest Dermatol 2005, 125: 545-553; Grether-Beck et al., Exp Dermatol 2008, 17: 771-779. Subsequently, the cells were then cultured in defined serum-free keratinocyte growth medium SFM (Invitrogen, Heidelberg, Germany) with the addition of bovine pituitary extract (Invitrogen, Heidelberg, Germany) and recombinant epidermal growth factor (Invitrogen, Heidelberg, Germany). The cells were incubated until passage 2 or 3 at 37° C. and 5% CO₂. For the test as to stimulation of the POMC gene expression, the cells were sown in 6-well plates and cultured to the point of subconfluence (maximum 70%).

Then, the medium was removed from the cells and replaced by fresh medium supplemented with 13-NODE. For this purpose, a 1000× stock solution of 10 000 μg/ml of 13-NODE dissolved in DMSO was used. End concentration of 13-HODE in the medium was 10 μg/ml (w/v), end concentration of DMSO in the medium 0.1% (v/v). Cultivations without 13-HODE, only with the vehicle DMSO were carried out as a control. All of the cultivations were carried out three times (3 biological replicates).

After cultivation for 24 hours, the cells were firstly washed in PBS buffer, and then admixed with fresh culture medium with 10 μg/ml 13-NODE. The cells were then cultured for a further 24 h until cell harvest.

The isolation of the whole-RNA and also the subsequent analysis of the gene expression by means of quantitative Real Time PCR (qRT-PCR) was carried out as described previously (Grether-Beck et al., J Invest Dermatol 2005, 125: 545-553; Grether-Beck et al., Exp Dermatol 2008, 17: 771-779). In summary, the whole-RNA was isolated by means of a RNeasy Total RNA Kit (Qiagen, Hilden, Germany). The RNA concentration was determined by means of photometric measurement at 260/280 nm (Biophotometer, Eppendorf, Germany). From each sample, 100 ng of whole-RNA were used for the cDNA synthesis, the Superscript®III-First Strand Synthesis System for RT-PCR (Invitrogen, Karlsruhe, Germany) being used. For the PCR reaction, the SYBR® Green PCR Master Mix was used in accordance with the manufacturer's instructions. Besides the POMC expression, the expression of the 18s rRNA gene was also quantitatively determined as a reference. In each case the following primer pairs were used for both target genes:

POMC:
(Seq ID No. 1)
5-′CGCTACGGCGGTTTCATG-3′
and
(Seq ID No. 2)
5-′TGATGATGGCGTTTTTGAACA-3′;
18S:
(Seq ID No. 3)
5′-GCCGCTAGAGGTGAAATTCTTG-3′
and
(Seq ID No. 4)
5′-CATTCTTGGCAAATGCTTTCG-3′.

The PCR reactions were carried out on an Opticon 1 (MJ Research, Waltham, Mass., USA). For all PCRs, double determinations of the biological triplicates were carried out, and for the evaluation averages of all measurements were calculated. The PCR protocol had the following profile: step 1), 10 minute activation of the hot start polymerase at 94° C.; step 2), 20 second denaturation at 95° C.; step 3), 20 second primer annealing at 55° C.; step 4), 30 second extension at 72° C. The cycle number of steps 2)-4) was 46. For the relative comparison of the gene expression, the 2(-delta delta C(T)) method was used (Livak KJ, and Schmittgen TD: Analysis of relative gene expression data using real time quantitative PCR and the 2 (-delta delta C(T)) method. Methods 2001, 25: 402-408).

The evaluation of the gene expression analysis showed that the treatment of the keratinocytes with 10 μg/ml 13-HODE increases the basal (not UV-stimulated) POMC expression 1.2-fold compared to the control treated with the vehicle (DMSO).

### Example 3

### Reduction in the UV-Stimulated Induction of Proinflammatory Marker Genes by 13-HODE

In the present example, the effect of 13-HODE on the expression of various proinflammatory marker genes in UV-B-stimulated keratinocytes (normal human epidermal keratinocytes, NHEKs) was investigated. The experimental procedure was largely identical to the procedure described in Example 1. Differences consisted merely in the selection of the gene-specific primers for carrying out the quantitative Real-Time PCR.

The following primer pairs were used in each case:

IL-6:
(Seq ID No. 5)
5′-AGCCGCCCCACACAGA-3′
and
(Seq ID No. 6)
5′-CCGTCGAGGATGTACCGAAT-3′;
IL-8:
(Seq ID No. 7)
5′-CTGGCCGTGGCTCTCTTG-3′
and
(Seq ID No. 8)
5′-TTAGCACTCCTTGGCAAAACTG-3′;
TNF-α:
(Seq ID No. 9)
5′-GGAGAAGGGTGACCGACTCA-3′
and
(Seq ID No. 10)
5′-TGCCCAGACTCGGCAAAG-3′;
COX-2:
(Seq ID No. 11)
5′-GAATCATTCACCAGGCAAATTG-3′
and
(Seq ID No. 12)
5′-TCTGTACTGCGGGTGGAACA-3′;
18S:
(Seq ID No. 3)
5′-GCCGCTAGAGGTGAAATTCTTG-3′
and
(Seq ID No. 4)
5′-CATTCTTGGCAAATGCTTTCG-3′.

The evaluation of the gene expression analysis showed that the exposure of the keratinocytes with UV light led to a clear induction of the expression of the investigated proinflammatory marker genes. Compared to the unexposed control, inductions of the gene expression by a factor of 10.1 (IL-6), 11.2 (IL-8), 7.4 (TNF-α) and 2.5 (COX2) were observed. Cells that were similarly exposed, but treated with 13-HODE in all cases had a considerably reduced expression level of the same marker genes. (FIGS. 3 to 6)

### Example 4

### Formulation Examples

### Example Formulation 1

### O/W Self-Tans with 13-HODE

| | Formulation | | | |
|---|---|---|---|---|
| | 1.1 | 1.2 | 1.3 | 1.4 |
| Polyglyceryl-3 | 3.0% | 3.0% | 3.0% | 3.0% |
| Methylglucose Distearate | | | | |
| Glyceryl Stearate | 2.0% | 2.0% | 2.0% | 2.0% |
| Stearyl Alcohol | 1.0% | 1.0% | 1.0% | 1.0% |
| Caprylic/Capric | 9.5% | 9.5% | 9.5% | 9.5% |
| Triglyceride | | | | |
| C12-15 Alkyl Benzoate | 9.5% | 9.5% | 9.5% | 9.5% |
| 13-HODE | — | 0.1% | 0.4% | 1.0% |
| Water | ad 100.0% | ad 100.0% | ad 100.0% | ad 100.0% |

### Example Formulation 2

### O/W Cream for Ethnic Skin (Self-Tans) with 13-HODE

| | Formulation | | | | |
|---|---|---|---|---|---|
| | 2.1 | 2.2 | 2.3 | 2.4 | 2.5 |
| Glyceryl Stearate | 1.5% | 1.5% | 1.5% | 1.5% | 1.5% |
| Citrate | | | | | |
| Cetearyl Alcohol | 3.0% | 3.0% | 3.0% | 3.0% | 3.0% |
| Glyceryl Stearate | 1.0% | 1.0% | 1.0% | 1.0% | 1.0% |
| Cetearyl | 11.0% | 11.0% | 11.0% | 11.0% | 11.0% |
| Ethylhexanoate | | | | | |
| Ethylhexyl Palmitate | 11.0% | 11.0% | 11.0% | 11.0% | 11.0% |
| Myristyl Myristate | 2.0% | 2.0% | 2.0% | 2.0% | 2.0% |
| 13-Hode | 0.5% | 0.5% | 0.5% | 0.5% | 0.5% |
| Glycerin; | — | — | — | — | 2.0% |
| Tetrapeptide-30 | | | | | |
| Glycerin | 5.0% | 5.0% | 5.0% | 5.0% | 5.0% |
| Water | ad | ad | ad 100% | ad | ad |
| | 100% | 100% | | 100% | 100% |
| Carbomer | 0.2% | 0.2% | 0.2% | 0.2% | 0.2% |
| Ethylhexyl Palmitate | 0.8% | 0.8% | 0.8% | 0.8% | 0.8% |
| Sodium Hydroxide | q.s. | q.s. | q.s. | q.s. | q.s. |
| (10% in water) | | | | | |
| Dihydroxyacetone | — | 1.0% | — | 1.0% | 1.0% |
| Erythrulose | — | — | 0.2% | 0.2% | 0.2% |
| Water | — | 5.0% | 5.0% | 5.0% | 5.0% |

### Example Formulation 3

### O/W Sunscreen Cream

| | Formulation | |
|---|---|---|
| | 3.1 | 3.2 |
| Polyglyceryl-3 Dicitrate/Stearate | 3.00% | 3.00% |
| Glyceryl Stearate | 1.25% | 1.25% |
| Cetearyl Alcohol | 1.25% | 1.25% |
| Diethylhexyl Carbonate | 2.50% | 2.50% |
| Oleyl Erucate | 2.00% | 2.00% |
| Myristyl Myristate | 1.20% | 1.20% |
| Caprylic/Capric Triglyceride | 3.00% | 3.00% |
| Bis-Ethylhexyloxyphenol Methoxyphenyl | 3.00% | 3.00% |
| Triazine | | |
| Octocrylene | 3.50% | 3.50% |
| Titanium Dioxide; Diethylhexyl | 4.40% | 4.40% |
| Carbonate; Polyglyceryl-6 | | |
| Polyhydroxystearate | | |
| 13-Hode | 1.00% | 1.00% |
| Glycerin | 3.00% | 3.00% |
| Water | ad 100.00% | ad 100.00% |
| Glycerin, Tetrapeptide-30 | 2.50% | — |
| Carbomer | 0.15% | 0.15% |
| Decyl Cocoate | 1.00% | 1.00% |
| Xanthan Gum | 0.20% | 0.20% |
| Sodium Hydroxide (10% in water) | 0.40% | 0.40% |

### Example Formulation 4

### Skin-Tightening O/W Sunscreen Lotion

| | Formulation |
|---|---|
| | 4.1 |
| Glyceryl Stearate Citrate | 2.00% |
| Cetearyl Alcohol | 1.00% |
| C12-15 Alkyl Benzoate | 5.00% |
| Triisostearin | 1.00% |
| Diethylhexyl Carbonate | 4.00% |
| Octocrylene | 6.00% |
| Bis-Ethylhexyloxyphenol Methoxyphenyl Triazine | 2.00% |
| Ethylhexyl Salicylate | 4.00% |
| Caprylic/Capric Triglyceride; Xymenynic Acid | 1.50% |
| Xanthan Gum | 0.20% |
| 13-Hode | 1.00% |
| Titanium Dioxide; Trimethoxycaprylylsilane; Glycerin | 3.00% |
| Glycerin | 3.00% |
| Water | ad 100.00% |
| Paraffinum Perliquidum | 0.80% |
| Carbomer | 0.20% |
| Sodium Hydroxide (10% in water) | 0.65% |

### Example Formulation 5

### Leave-in Conditioner

| | Formulation |
|---|---|
| | 5.1 |
| Hydrolyzed Keratin | 2.50% |
| Water | ad 100.00% |
| Sodium Lactate; Sodium PCA; Glycine; Fructose; Urea; | 2.00% |
| Niacinamide; Inositol; Sodium Benzoate; Lactic Acid | |
| PEG-40 Hydrogenated Castor Oil | 1.00% |
| Quaternium-80 | 1.50% |
| Ethanol | 15.00% |
| PVP/VA Copolymer | 4.00% |
| 13-Hode | 0.10% |
| Cocamidopropyl Betaine | 4.00% |
| Citric Acid (30%) | 3.00% |

### Example Formulation 6

### Leave-in Conditioning Spray

| | Formulation |
|---|---|
| | 6.1 |
| Laureth-4 | 0.5% |
| PEG-40 Hydrogenated Castor Oil | 0.5% |
| 13-Hode | 0.1% |
| Quaternium-80 | 0.4% |
| Dimethicone Propyl PG-Betaine | 0.6% |
| Cetrimonium Chloride | 0.8% |
| Water | ad 100.0% |
| Creatine | 0.5% |
| Ethanol | 15.0% |
| PVP/VA Copolymer | 4.0% |
| Sodium Hydroxide (10% in water) | 1.2% |

### Example Formulation 7

### Permanent Hair Colorant

| | Formulation |
|---|---|
| | 7.1 |
| Cetearyl Alcohol | 7.0% |
| Lanolin | 1.5% |
| Ceteareth-20 | 1.5% |
| Laneth-5 | 1.0% |
| Polyquaternium-10 | 1.0% |
| Ammonium sulphate | 0.5% |
| Sodium sulphite | 0.5% |
| Disodium EDTA | 0.1% |
| p-Toluene-2,5-diamine, sulphate | 0.85% |
| 4-Amino-2-Hydroxytoluene | 0.17% |
| 5-Amino-6-Chloro-o-Cresol | 0.36% |
| 4-Chlororesorcinol | 0.07% |
| Ammonium hydroxide (25%) | 7.0% |
| 13-Hode | 0.1% |
| Water | ad 100.00% |

Prior to use, mix 1:1 with developer emulsion

### Example Formulation 7A

### Developer Emulsion for Formulation 7

| | Formulation |
|---|---|
| | 8.1 |
| Cetyl Alcohol | 3.0% |
| Ceteareth-20 | 1.0% |
| Sodium Cetearyl Sulphate | 1.0% |
| Hydrogen Peroxide (50% strength) | 12.0% |
| Phosphoric Acid (85%) | 0.5% |
| Sodium Stannate | 0.1% |
| Water | ad 100.00% |

### Example Formulation 8

### Tinting Lotion (Semi-Permanent)

| | Formulation |
|---|---|
| | 9.1 |
| Cetearyl alcohol | 3.0% |
| Parafinium liquidum | 1.0% |
| PEG-40 Hydrogenated Castor Oil | 0.3% |
| Cetrimonium Chloride | 4.0% |
| 2,6-Dihydroxy ethylaminotoluene | 0.25% |
| HC Red No. 3 | 0.25% |
| HC Yellow No. 2 | 0.3% |
| 13-Hode | 0.1% |
| Water | ad 100% |

### Example 5

### Human In Vivo Study for Demonstrating the Maintenance of the Skin Tan by a Cosmetic Formulation Comprising 13-HODE

In order to test to what extent 13-HODE has an influence on maintaining the skin tan, the maintenance of the summer tan under the influence of 13-HODE in test formulations was investigated in a clinical study. The study was carried out over a period of 8 weeks from mid-September to mid-November in Germany. The subject of the investigation was the skin colour by means of colorimetric measurements using a Skin-Colorimeter® CL 400 from Courage+Khazaka electronic GmbH, Cologne, Germany. The measurements were made in each case on the inside of the left and right forearm on a defined area measuring 3 cm×3 cm at a distance of 11-14 cm from the wrist. The average value, which resulted from the individual values of six to nine repeat measurements, was registered. 39 people aged 21-55 years participated, of which 28 were female and 11 male. In each case, the starting value of the skin colour TO was determined prior to the treatment and the value T8 was determined after treatment for 56 days with test formulations. The application frequency was twice daily, morning and evening. The test persons were given one formulation for the left arm and one for the right arm (formulation A, B, C, D; as per the table below).

| | | Test formulation | | | |
|---|---|---|---|---|---|
| Product | INCI | A | B | C | D |
| TEGO Care 450 | Polyglyceryl-3 | 3.0 | 3.0 | 3.0 | 3.0 |
| | Methylglucose Distearate | | | | |
| TEGIN M Pellets | Glyceryl Stearate | 2.0 | 2.0 | 2.0 | 2.0 |
| TEGO Alkanol | Stearyl Alcohol | 1.0 | 1.0 | 1.0 | 1.0 |
| 18 | | | | | |
| TEGOSOFT CT | Caprylic/Capric | 9.5 | 9.5 | 9.5 | 9.5 |
| | Triglyceride | | | | |
| TEGOSOFT TN | C12-15 Alkyl Benzoate | 9.5 | 9.5 | 9.5 | 9.5 |
| 13-HODE | 13-HODE | — | 0.1 | 0.4 | 1.0 |
| Water | Water | ad | ad | ad | ad |
| | | 100.0 | 100.0 | 100.0 | 100.0 |
| Euxyl K 220 | Water, | 0.1 | 0.1 | 0.1 | 0.8 |
| | Methylisothiazolinone, | | | | |
| | Ethylhexylglycerin | | | | |
| Perfume | Perfume | 0.4 | 0.4 | 0.4 | 0.4 |

Data in percent by mass.

As the human in vivo study showed, over the period of eight weeks, the decrease in the blue-yellow parameter b* and also the increase in the parameter ITA° (28°≧ITA°>10° matt/light brown, 41°≧ITA°>28° intermediate, 55°≧ITA°>41° light, ITA°>55° very light) of the skin colour could be reduced to differing degrees by using increasing concentrations of 13-NODE in the formulation. The results are shown graphically in FIGS. 7 and 8.

These results show that a reduction in the lightening of the skin colour can be achieved on account of using 13-HODE.

1. A method for tanning human skin and/or for maintaining an existing skin tan, the method comprising applying 13-hydroxy-9,11-octadecadienoic acid to human skin.

2. (canceled)

3. The method of claim 1, wherein said 13-hydroxy-9,11-octadecadienoic acid is a component in a cosmetic formulation.

4. The method of claim 1, wherein said tanning is achieved without exposing the skin to UV radiation.

5. The method of claim 1, wherein said human skin is exposed to UV light after 13-hydroxy-9,11-octadecadienoic acid and/or a formulation comprising 13-hydroxy-9,11-octadecadienoic acid is applied to the skin.

6. The method of claim 1, wherein uneven coloration induced by dihydroxyacetone and/or erythrulose is corrected by applying said 13-hydroxy-9,11-octadecadienoic acid and/or a formulation comprising 13-hydroxy-9,11-octadecadienoic acid to skin having an uneven coloration as a result of treatment with dihydroxyacetone and/or erythrulose.

7. A method for coloring human hair, the method comprising applying 13-hydroxy-9,11-octadecadienoic acid to the scalp.

8. The method according to claim 1, wherein said 13-hydroxy-9,11-octadecadienoic acid is applied repeatedly to the skin.

9. The method of claim 1, wherein said method also treats skin having polymorphous photodermatosis, and/or prevents the skin from polymorphous photodermatosis.

10. The method of claim 1, wherein said 13-Hydroxy-9,11-octadecadienoic acid has a 9-Z 11-E isomeric configuration.

11. The method of claim 1, wherein said 13-Hydroxy-9,11-octadecadienoic acid has an S stereoconfiguration at the C13 carbon atom.

12. The method of claim 1, wherein said 13-Hydroxy-9,11-octadecadienoic acid has a 9-Z 11-E isomeric configuration and an S stereoconfiguration at the C13 carbon atom.

13. The method of claim 3, wherein said cosmetic formulation comprises said 13-Hydroxy-9,11-octadecadienoic acid in an amount of 0.00001 to 1 percent by mass based on the total mass of the cosmetic formulation.

14. The method of claim 3, wherein said cosmetic formulation comprises said 13-Hydroxy-9,11-octadecadienoic acid in an amount of 0.00005 to 0.5 percent by mass based on the total mass of the cosmetic formulation.

15. The method of claim 3, wherein said cosmetic formulation comprises said 13-Hydroxy-9,11-octadecadienoic acid in an amount of 0.0001 to 0.1 percent by mass based on the total mass of the cosmetic formulation.

16. The method of claim 3, wherein said cosmetic formulation is a sunscreen formulation.

17. The method of claim 16, wherein said sunscreen formulation comprises at least one UV radiation-absorbing substance.

18. The method of claim 17, wherein said at least one UV radiation-absorbing substance is selected from cinnamic esters, ester of salicylic acid, benzophenone, and finely dispersed metal oxides.

19. The method of claim 7, further comprising restoring original hair color in hair that has become gray.


SEQUENCE LISTING (12 sequences)

SEQ ID NO: 1 - DNA, 18 bp, Artificial Sequence
  Synthetic Oligonucleotide
cgctacggcggtttcatg

SEQ ID NO: 2 - DNA, 21 bp, Artificial Sequence
  Synthetic Oligonucleotide
tgatgatggcgtttttgaaca

SEQ ID NO: 3 - DNA, 22 bp, Artificial Sequence
  Synthetic Oligonucleotide
gccgctagaggtgaaattcttg

SEQ ID NO: 4 - DNA, 21 bp, Artificial Sequence
  Synthetic Oligonucleotide
cattcttggcaaatgctttcg

SEQ ID NO: 5 - DNA, 16 bp, Artificial Sequence
  Synthetic Oligonucleotide
agccgccccacacaga

SEQ ID NO: 6 - DNA, 20 bp, Artificial Sequence
  Synthetic Oligonucleotide
ccgtcgaggatgtaccgaat

SEQ ID NO: 7 - DNA, 18 bp, Artificial Sequence
  Synthetic Oligonucleotide
ctggccgtggctctcttg

SEQ ID NO: 8 - DNA, 22 bp, Artificial Sequence
  Synthetic Oligonucleotide
ttagcactccttggcaaaactg

SEQ ID NO: 9 - DNA, 20 bp, Artificial Sequence
  Synthetic Oligonucleotide
ggagaagggtgaccgactca

SEQ ID NO: 10 - DNA, 18 bp, Artificial Sequence
  Synthetic Oligonucleotide
tgcccagactcggcaaag

SEQ ID NO: 11 - DNA, 22 bp, Artificial Sequence
  Synthetic Oligonucleotide
gaatcattcaccaggcaaattg

SEQ ID NO: 12 - DNA, 20 bp, Artificial Sequence
  Synthetic Oligonucleotide
tctgtactgcgggtggaaca